(19) United States
(12) Patent Application Publication
(10) Pub. No.: US 2015/0315149 A1
(43) Pub. Date: Nov. 5, 2015

(54) METHODS OF MAKING NETUPITANT AND INTERMEDIATES THEREOF

(71) Applicant: Apicore US LLC, Somerset, NJ (US)

(72) Inventors: Ravishanker Kovi, Monroe, NJ (US); Jayaraman Kannapan, Gujarat (IN); Sanjay F. Thakor, Gujarat (IN); Ashish Naik, Piscataway, NJ (US)

(21) Appl. No.: 14/703,178

(22) Filed: May 4, 2015

Related U.S. Application Data
(60) Provisional application No. 61/988,434, filed on May 5, 2014.
(60) Provisional application No. 62/045,884, filed on Sep. 4, 2014.

(51) Int. Cl. C07D 213/75; C07F 5/02

(52) U.S. Cl. CPC C07D 213/75; C07F 5/025

(57) ABSTRACT

Methods are disclosed for the preparation of netupitant and pharmaceutically acceptable salts thereof which are novel, easily reproducible, environmentally safe and cost effective. The methods may employ inexpensive starting materials and the preparation processes for intermediates are simple and highly reproducible. Novel intermediates for the preparation of netupitant and pharmaceutically acceptable salts thereof are also disclosed. Amorphous netupitant and methods of making same are disclosed.

### CROSS-REFERENCE TO RELATED APPLICATIONS

This application claims the benefit of U.S. Provisional Patent Application No. 61/988,434 filed May 5, 2014 and U.S. Provisional Patent Application No. 62/045,884 filed Sep. 4, 2014, the entireties of which are incorporated by reference herein.

### FIELD OF INVENTION

The present invention describes a novel method of making the NK₁ receptor antagonist netupitant.

### BACKGROUND

Netupitant hydrochloride, 2-(3,5-bis(trifluoromethyl)phenyl)-N,2-dimethyl-N-(6-(4-methylpiperazin-1-yl)-4-(o-tolyl)pyridin-3-yl)propanamide dihydrochloride of formula (I), is a potent and selective NK1 receptor antagonist (NK1 RA).

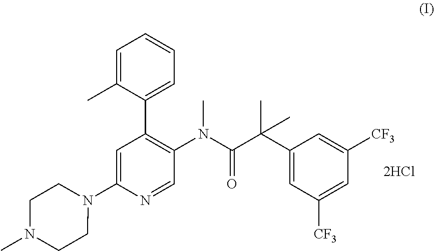 [SMILES: Cc1ccccc1-c1cc(N2CCN(C)CC2)ncc1N(C)C(=O)C(C)(C)c1cc(C(F)(F)F)cc(C(F)(F)F)c1]

### SUMMARY OF THE INVENTION

NK1 RAs are commonly coadministered with a 5-HT3 RA such as palonosetron to prevent chemotherapy-induced-nausea and vomiting (CINV). Netupitant is both a substrate for and a moderate inhibitor of CYP3A4.

U.S. Pat. No. 6,297,375 discloses a method of preparation of formula (I) based on the condensation of N-methyl-6-(4-methylpiperazin-1-yl)-4-(o-tolyl)pyridin-3-amine of formula (IG)

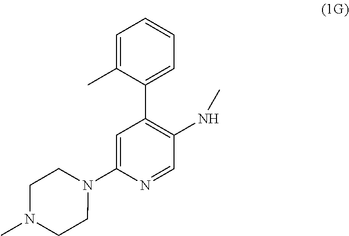

with 2-(3,5-bis(trifluoromethyl)phenyl)-2-methylpropanoyl chloride (formula 1H).

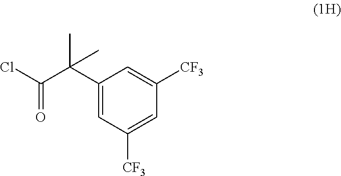 [SMILES: CC(C)(C(=O)Cl)c1cc(C(F)(F)F)cc(C(F)(F)F)c1]

According to this patent, the preparation of netupitant requires the following route of synthesis starting with 2-chloro-5-aminopyridine:

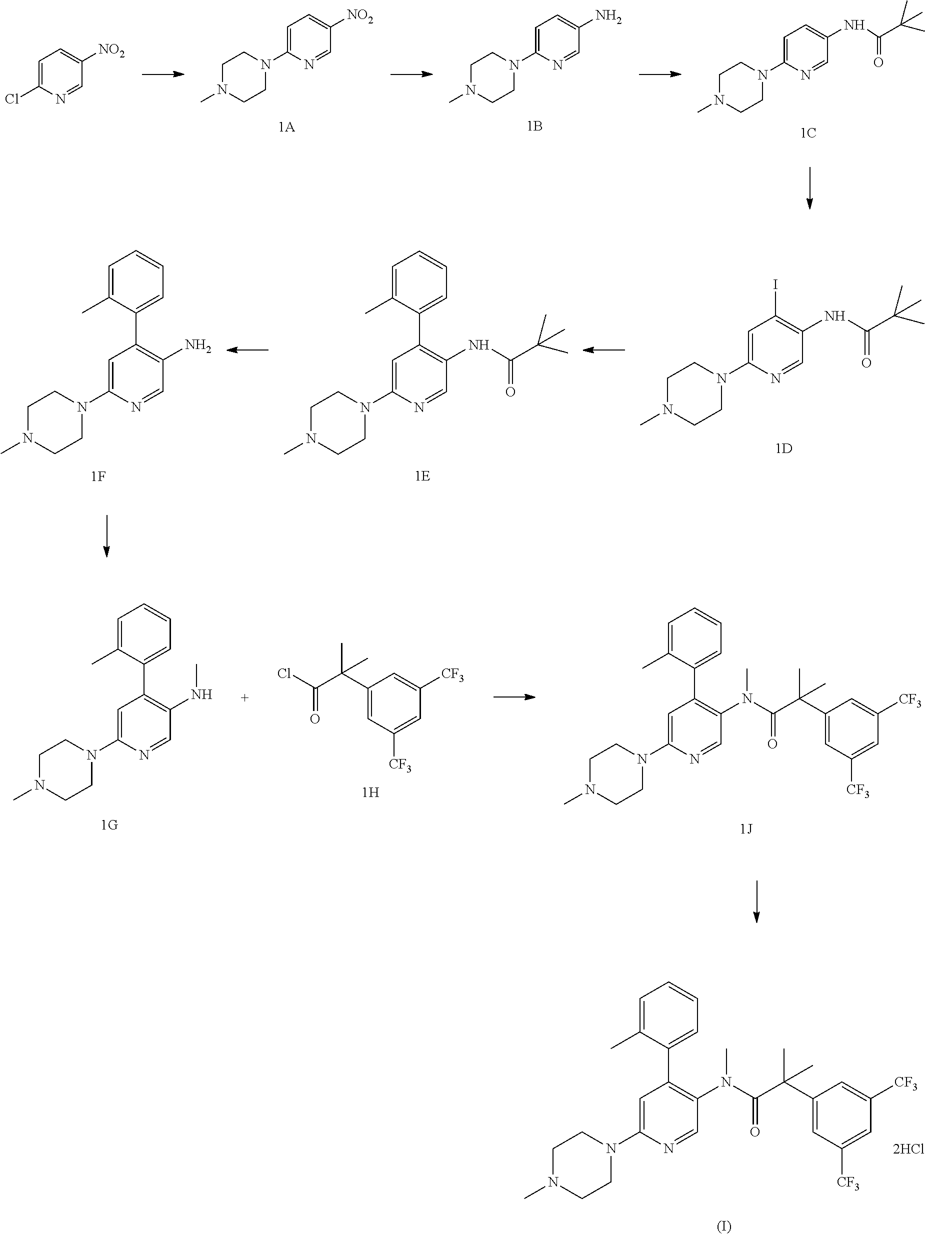

The above-referenced process involves a multistep synthesis of making the product with highly hazardous reactions, work up and isolation. The process involves the use of intermediates which are extremely difficult to scale up and are very low yielding. For example, the preparation of a key intermediate-(6-(4-methylpiperazin-1-yl)-4-(o-tolyl)pyridin-3-yl) pivalamide of formula (1C)

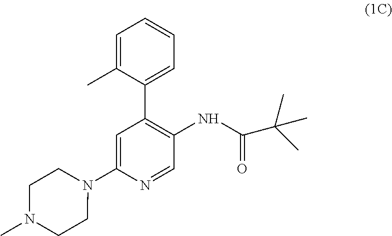 [SMILES: Cc1ccccc1-c1cc(N2CCN(C)CC2)ncc1NC(=O)C(C)(C)C]

according to U.S. Pat. No. 6,297,375 involves a cumbersome process of making the iodo compound using iodine and the highly hazardous reagent butyl lithium at a very low temp of −70° C. The yields of the process are very low, and the process involves a long column purification and very low temperature conditions which are not easy for scale up and commercial operations.

By way of further example, the preparation of another intermediate N-methyl-6-(4-methylpiperazin-1-yl)-4-(o-tolyl)pyridin-3-amine

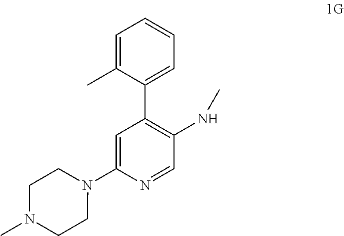

involves the use of lithium aluminium hydride reduction and column isolation. Reducing agents like lithium aluminium are highly hazardous in large scale operations.

Thus, there is a need for alternative methods which overcome the above-referenced drawbacks

The presently disclosed subject matter provides methods which address the foregoing drawbacks. In accordance with one or more embodiments, methods are disclosed for the preparation of 2-(3,5-bis(trifluoromethyl)phenyl)-N,2-dimethyl-N-(6-(4-methylpiperazin-1-yl)-4-(o-tolyl)pyridin-3-yl)propanamide dihydrochloride of formula (I) which are novel, easily reproducible, environmentally safe and cost effective. The methods may employ inexpensive starting materials and the preparation processes for intermediates are simple and highly reproducible. The methods do not involve any hazardous reagents or difficult reaction conditions and are free from column isolation procedures. Novel intermediates for the preparation of netupitant and pharmaceutically acceptable salts thereof are also disclosed.

In accordance with one embodiment a method of making 2-(3,5-bis(trifluoromethyl)phenyl)-N,2-dimethyl-N-(6-(4-methylpiperazin-1-yl)-4-(o-tolyl)pyridin-3-yl)propanamide dihydrochloride of formula (I) includes a reaction of a novel intermediate 2-(3,5-bis(trifluoromethyl)phenyl)-N,2-dimethyl-N-(6-(4-methylpiperazin-1-yl)-4-(4,4,5,5-tetramethyl-1,3,2-dioxaborolan-2-yl)pyridin-3-yl)propanamide of formula 2H and a 2-halo-substituted toluene of the formula 2K wherein Y is Cl, Br or I, such as 1-iodo-2-methylbenzene.

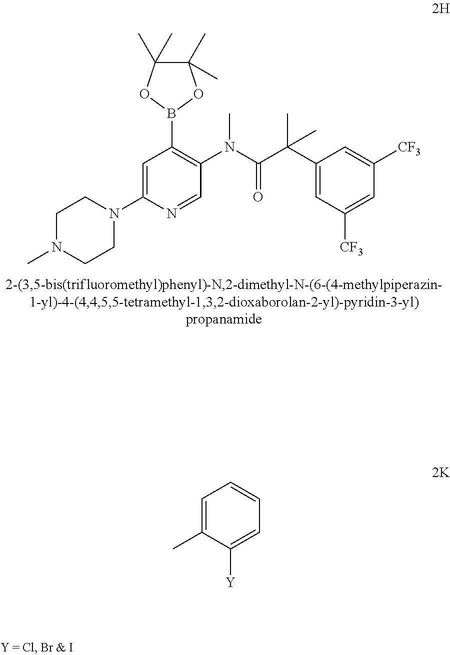

In another embodiment a method of making the compound of formula I includes coupling a compound of the formula 2J wherein X is an alcohol protecting group such as but not limited to Cl, Br, OCH₃, OTf, OBz, o-pivaloyl and/or another suitable alcohol protecting group, with O-tolyl boronic acid of the formula 2L. For example a suitable compound of the formula 2J may be 2-(3,5-bis(trifluoromethyl)phenyl)-N-(4-chloro-6-(4-methylpiperazin-1-yl)pyridin-3-yl)-N,2-dimethylpropanamide.

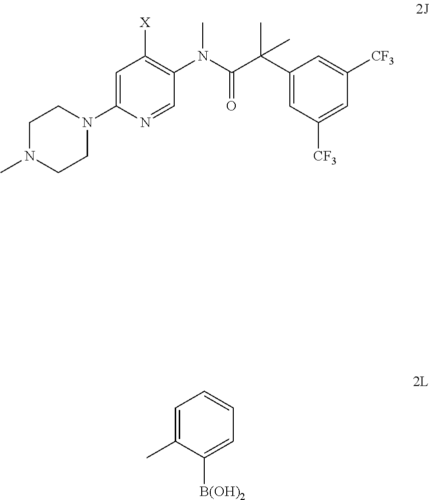

In further embodiments a simple and commercially scalable process of making a compound of formula 2J is disclosed which involves condensing 4-chloro-6-(4-methylpiperazin-1-yl)pyridin-3-amine with 2-(3,5-bis(trifluoromethyl)phenyl)-2-methylpropanoyl chloride in the presence of an organic base such as diisopropyl ethylamine, triethylamine and an organic base such as pyridine.

According to embodiments disclosed herein, the preparation of formula (I) 2-(3,5-bis(trifluoromethyl)phenyl)-N,2-dimethyl-N-(6-(4-methylpiperazin-1-yl)-4-(o-tolyl)pyridin-3-yl)propanamide dihydrochloride may start with a very simple and a commercial raw material such as 4-methoxy-3-nitro pyridine. For example, SCHEME-II, below, starts from 4-methoxy-3-nitro pyridine and clearly highlights the advantages over the methods in U.S. Pat. No. 6,297,375.

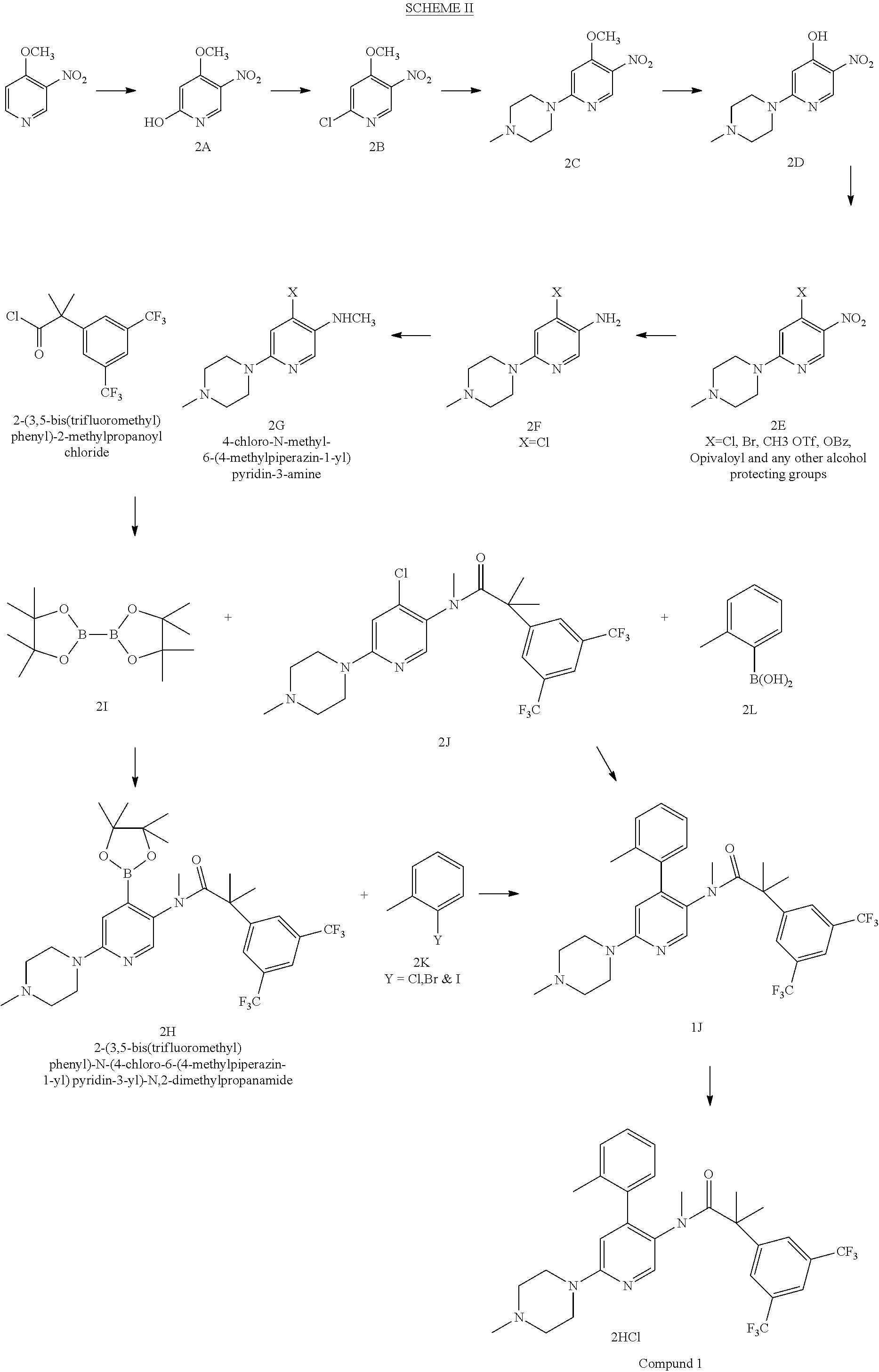

In another embodiment a method of making netupitant and pharmaceutically acceptable salts thereof is provided according to SCHEME III.

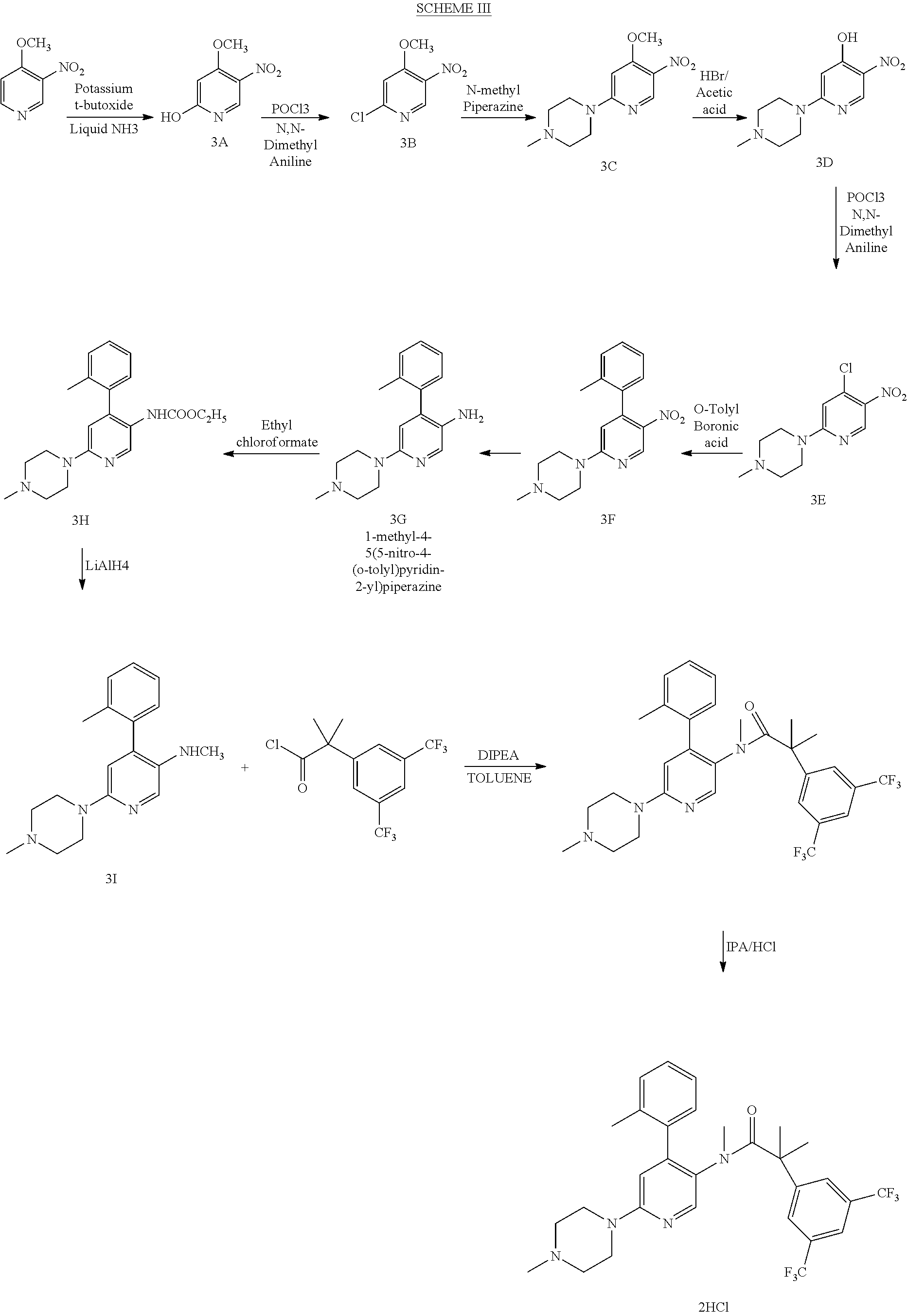

Methods in accordance with SCHEME III employ a novel intermediate having the formula 3H.

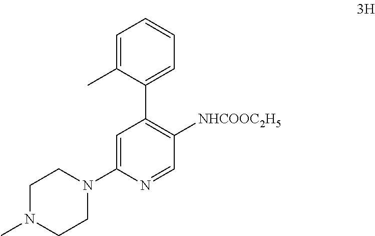

In accordance with further embodiments amorphous netupitant and methods of making same are disclosed.

### BRIEF DESCRIPTION OF THE DRAWINGS

So that those having ordinary skill in the art will have a better understanding of how to make and use the disclosed systems and methods, reference is made to the accompanying figures wherein:

FIG. 1 is a graphical depiction of XRD data in accordance with an embodiment of the present invention; and

FIG. 2 is a graphical depiction of XRD data in accordance with an embodiment of the present invention.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

The present inventors have found that netupitant, and salts thereof (such as dihydrochloride) can be prepared in high purity and high yield by methods disclosed herein such as SCHEME II and SCHEME III disclosed above.

In one aspect, the present invention describes a process of making 2-(3,5-bis(trifluoromethyl)phenyl)-N,2-dimethyl-N-(6-(4-methylpiperazin-1-yl)-4-(o-tolyl)pyridin-3-yl)propanamide dihydrochloride of the formula I by coupling a 2-halo substituted toluene compound such as but not limited to an intermediate of the formula 2K wherein Y is Cl, Br or I with an intermediate of the formula 2H.

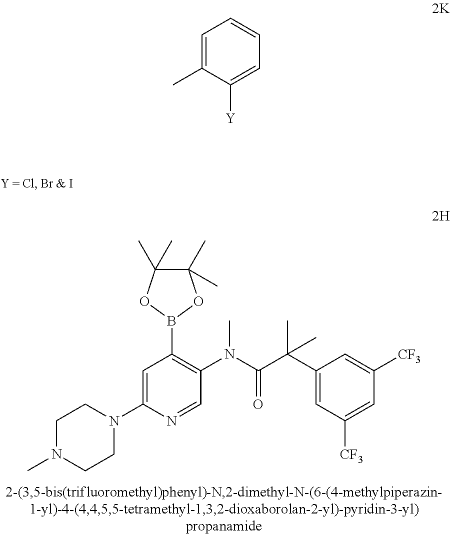

In another embodiment, a process is provided for making netupitant dihydrochloride of the formula I by coupling an intermediate of the formula 2L with an intermediate of the formula 2J wherein X is Cl, Br, OCH₃, trifluoromethanesulfonate (OTf), O-benzyl (OBz), (CH3)₃CCOCl (o-pivaloyl) and/or another suitable alcohol protecting group.

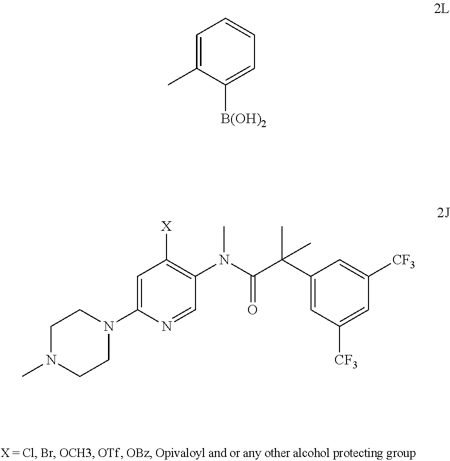

The present inventors have found that the above mentioned compounds of formulae 2J & 2H can be very efficiently achieved employing commercially available 4-methoxy-2-nitro pyridine as a very basic starting material. Thus, in another aspect, the present invention relates to novel methods of preparation of the intermediates 2J and 2H from 4-methoxy-3-nitro pyridine.

The preparation of these intermediates may start with hydroxy introduction in the 4-methoxy-3-nitro pyridine. The hydroxy introduction results in the formation of 2-hydroxy-4-methoxy-5 nitro pyridine. The hydroxy substitution may be carried out using t-butyl hydro peroxide and potassium tert butoxide in tetrahydrofuran solvent medium with liquor ammonia. Typically, the reaction may be carried out anywhere in the temperature range of −70° C. to 0° C. In one embodiment the reaction may be carried out between −30° C. to −40° C.

Solvents that may be used for the above reaction include 1,4-dioxane and tetrahydrofuran. In one embodiment tetrahydrofuran is the solvent used for this reaction.

Preparation of 2-chloro-4-methoxy-5-nitro pyridine from 2-hydroxy-4-methoxy-5 nitro pyridine may be achieved using phosphorous oxychloride, phosphorous chloride, thionyl chloride or oxalyl chloride. In one embodiment phosphorous oxychloride is the reagent used for this conversion.

Bases which may be employed for this conversion include pyridine, dimethyl aniline, diethyl aniline, diisopropyl ethylamine and related organic bases. In one embodiment the reaction is carried out in the presence of diethyl or dimethyl aniline.

The reaction temperature for the preparation of 2-chloro-4-methoxy-5-nitro pyridine may be from about 100-120° C. and in one embodiment, from about 110-115° C.

The preparation of 1-(4-methoxy-5-nitropyridin-2-yl)-4-methylpiperazine from 2-chloro-4-methoxy-5-nitro pyridine may be carried out using N-methyl piperazine, in the presence of a solvent such as tetrahydrofuran, toluene, or dichloromethane.

Generally, the reaction temperature for the N-methyl piperazine attachment to the 2-chloro-4-methoxy-5-nitro pyridine may be from about 25° C. to 110° C. and in one embodiment, from about 25° C. to 35° C.

The preparation of 2-(4-methylpiperazin-1-yl)-5-nitropyridin-4-ol from 1-(4-methoxy-5-nitropyridin-2-yl)-4-methylpiperazine may be carried out by the demethylation of the 4-methoxy group of 1-(4-methoxy-5-nitropyridin-2-yl)-4-methylpiperazine. The demethylation may be effected in the presence of reagents such as HBr in acetic acid, HBr in water, boron tribromide, boron trichloride, aluminium chloride, etc. In one embodiment HBr in acetic acid is employed as the reagent for this conversion.

The temperature employed for the demethylation may be from about 25° C. to about 125° C. and in one embodiment, from about 105° C. to about 115° C.

The preparation of 1-(4-chloro-5-nitropyridin-2-yl)-4-methylpiperazine from 2-(4-methylpiperazin-1-yl)-5-nitropyridin-4-ol may be carried out using the above mentioned chlorination process which may involve phosphorous oxychloride and dimethyl aniline.

The reduction of 1-(4-chloro-5-nitropyridin-2-yl)-4-methylpiperazine to 4-chloro-6-(4-methylpiperazin-1-yl)pyridin-3-amine may be carried out in the presence of hydrogen with catalysts such as palladium/carbon, Raney nickel, etc. The reduction may employ other conditions such as palladium carbon/ammonium formate and palladium carbon ammonium acetate. Other reduction conditions which may be employed for the above conversion include tin/HCl, Fe/HCl and zinc/HCl conditions. The reduction may employ solvents such as methanol, ethanol, acetic acid, ethyl acetate and combinations of these solvents. In one embodiment iron/acetic acid is employed. The reduction may be carried out at a temperature of from about 25° C. to about 125° C. In one embodiment iron/acetic acid is employed at from about 60° C. to about 70° C.

The preparation of 4-chloro-N-methyl-6-(4-methylpiperazin-1-yl)pyridin-3-amine from 4-chloro-6-(4-methylpiperazin-1-yl)pyridin-3-amine may be carried out using different methylation processes. The N-methylation of amine may employ different conditions such as formic acid/sodium borohydride, protection of the amine with protection agents such as BOC, Fmoc followed by methylation with methyl iodide and subsequent deprotection of the protecting groups.

Monomethylation may be effected with the reaction of 4-chloro-6-(4-methylpiperazin-1-yl)pyridin-3-amine with trimethyl orthoformate and subsequent reduction with lithium aluminium hydride.

Preparation of a compound of the formula 2J, such as for example 2-(3,5-bis(trifluoromethyl)phenyl)-N-(4-chloro-6-(4-methylpiperazin-1-yl)pyridin-3-yl)-N,2-dimethylpropanamide, may be carried out by condensing 4-chloro-6-(4-methylpiperazin-1-yl)pyridin-3-amine with 2-(3,5-bis(trifluoromethyl)phenyl)-2-methylpropanoyl chloride in the presence of an organic base such as diisopropyl ethylamine, triethylamine and an organic base such as pyridine. The reaction may be carried out in a solvent such as dichloromethane, dimethyl formamide, toluene, acetonitrile, dimethyl acetamide, dimethyl sulfoxide, etc. In one embodiment the solvent is toluene. The reaction may be carried out in the range of from about 45° C. to about 140° C. In one embodiment the reaction is carried out in the range of from about 100° C. to about 115° C.

In accordance with one or more embodiments, preparation of netupitant, 2-(3,5-bis(trifluoromethyl)phenyl)-N,2-dimethyl-N-(6-(4-methylpiperazin-1-yl)-4-(o-tolyl)pyridin-3-yl)propanamide, is carried out by coupling a compound of the formula 2J such as but not limited to 2-(3,5-bis(trifluoromethyl)phenyl)-N-(4-chloro-6-(4-methylpiperazin-1-yl)pyridin-3-yl)-N,2-dimethylpropanamide and O-tolyl boronic acid. The coupling of 2-(3,5-bis(trifluoromethyl)phenyl)-N-(4-chloro-6-(4-methylpiperazin-1-yl)pyridin-3-yl)-N,2-dimethylpropanamide (2J) and O-tolyl boronic acid may be carried out in the presence of a catalyst such as tetrakis (triphenyl phosphine) palladium (0) and bis(triphenylphosphine)palladium (II) dichloride. In one embodiment the catalyst is tetrakis (triphenyl phosphine) palladium (0). The solvent employed in the coupling may be isopropanol, methanol, n-butanol or toluene. In one embodiment the solvent is toluene. The reaction temperature employed for the above conversion may be from about 80° C. to about 120° C. In one embodiment the temperature range is from about 80° C. to about 90° C.

The formation of the dihydrochloride may be achieved by addition of concentrated hydrochloric acid to the 2-(3,5-bis(trifluoromethyl)phenyl)-N,2-dimethyl-N-(6-(4-methylpiperazin-1-yl)-4-(o-tolyl)pyridin-3-yl)propanamide free base by dissolving the base in a solvent such as diisopropylether, acetone, or ethyl acetate at from about 0° C. to 5° C. In one embodiment the solvent is diisopropylether.

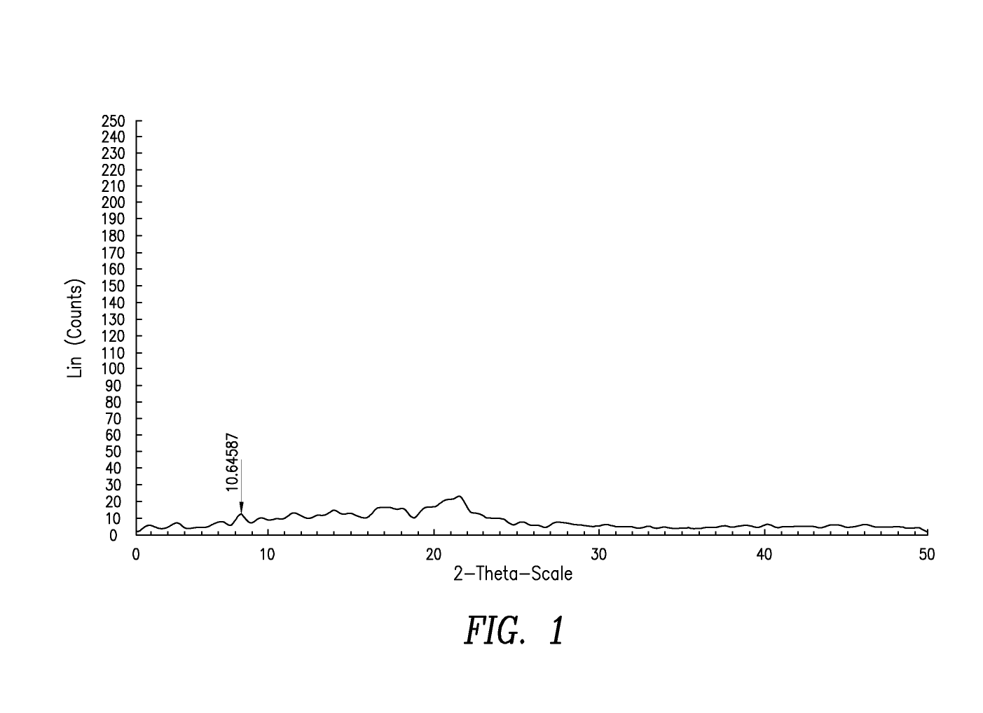

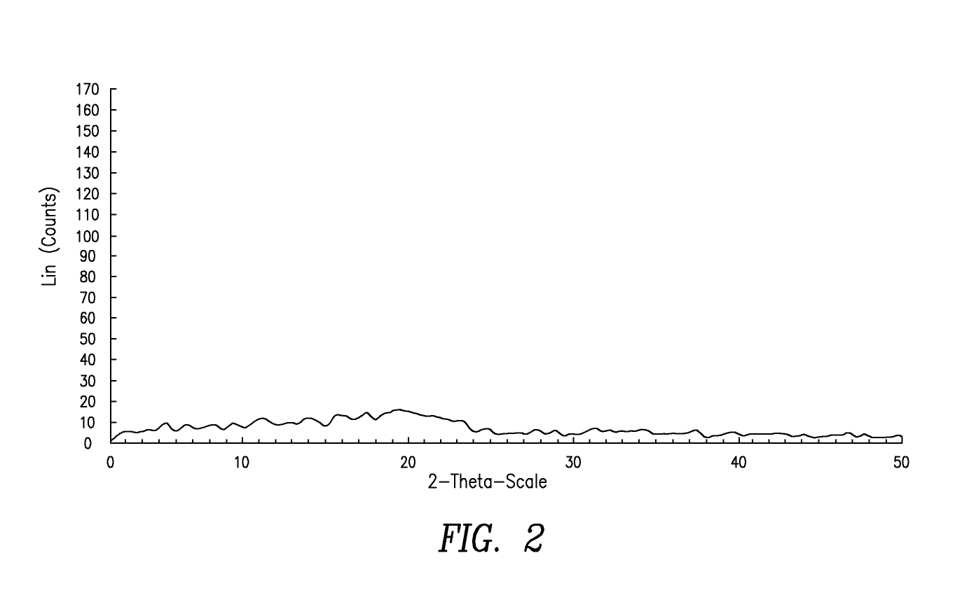

Now referring to FIGS. 1 and 2, amorphous netupitant was produced using povidone as further described hereinbelow.

The preparation processes for netupitant and pharmaceutically acceptable salts thereof disclosed herein involve novel intermediates which are easily prepared and can be scaled up without difficulties. The intermediates are solid in nature and isolated by conventional isolation techniques, avoiding column purification. As such, the methods have commercial-scale applicability. Dangerous reagents such as n-butyl lithium are avoided, as are very low temperature conditions (which are commercially unfavourable) for iodination of pyridine ring and subsequent biphenyl formation. The presently disclosed methods also avoid the application of commercially difficult reaction conditions such as Grignard-type reactions to introduce the biphenyl group. Process times, for example the cycle time involved in making the intermediates and the final product, are drastically reduced over prior art methods, making the processes commercially desirable. The processes are environmentally friendly, industrially applicable, economical and free from hazardous reagents and extremely difficult reaction conditions.

### EXPERIMENTS/EXAMPLES

### Scheme II Experiments/Examples

### Example 1

### Preparation of 2-hydroxy-4-methoxy-5-nitro pyridine(4-methoxy-5-nitropyridin-2-ol)

Charged ammonia gas at −70° C. to a solution of THF (1800 ml). After achieving 1.0 Kg, stopped the gas flow. Charged potassium tert butoxide (182 gms). Temperature was raised to −30° C., charged a solution of 4-methoxy-3-nitro pyridine (100 g) dissolved in THF (250 ml) and tert butyl hydro peroxide (144 ml) to the above mixture at −30 to −25° C. After addition, continued the reaction for 1.0 hr. Saturated ammonium chloride solution (450 ml) was cautiously added and the mixture was allowed to warm to room temperature. The ammonia was evaporated and the residue diluted with water (500 mL). The resulting solid was collected, washed with water and dried to give 2-hydroxy-4-methoxy-5-nitro pyridine(4-methoxy-5-nitropyridin-2-ol) (90.0 g).

### Example 2

### Preparation of 2-chloro-4-methoxy-5-nitro pyridine

Charged 2-hydroxy-4-methoxy-5-nitro pyridine (100 g) and dimethylaniline (92 mL) at 10° C.- to 20° C. Added phosphorus oxychloride (300 mL) slowly at 10° C. to 20° C. Refluxed for 2.0 hrs. Cooled to room temperature and dumped reaction mixture in ice and extracted in ethyl acetate after adjusting the pH to around 10 to 12. Treated the organic layer with brine solution and dried over sodium sulphate. Distilled off under reduced pressure to give residue which on crystallisation with diisopropyl ether gave 2-chloro-4-methoxy-5-nitro pyridine (70.0 g).

### Example 3

### Preparation of 1-(4-methoxy-5-nitropyridin-2-yl)-4-methylpiperazine

Charged 2-chloro-4-methoxy-5-nitro pyridine (100 g) N-methyl piperazine (175 mL) and tetrahydrofuran (1200 mL) and stirred for 10 hrs at room temperature. Charged reaction mixture in 720 mL of water and extracted in ethyl acetate. Organic layer washed with brine solution and treated with sodium sulphate. Distilled off organic layer under reduced pressure; crystallisation with diisopropyl ether gave 1-(4-methoxy-5-nitropyridin-2-yl)-4-methylpiperazine (115.0 gms).

### Example 4

### Preparation of 2-(4-methylpiperazin-1-yl)-5-nitropyridin-4-ol

Charged 1-(4-methoxy-5-nitropyridin-2-yl)-4-methylpiperazine (100 g), 38% HBr in acetic acid (500 mL) and acetic acid (500 mL). Stirred for 8 hrs. at 105° C. Cooled to room temperature, filtered the solid and washed with 100 mL acetic acid. Dried the solid at 70° C. for 10 hrs to give 145 g of 2-(4-methylpiperazin-1-yl)-5-nitropyridin-4-ol.

### Example 5

### Preparation of 1-(4-chloro-5-nitropyridin-2-yl)-4-methylpiperazine

Charged 2-(4-methylpiperazin-1-yl)-5-nitropyridin-4-ol (100.0 g) and N,N-dimethylaniline (92 mL) at 10° C. to 20° C. Added phosphorus oxychloride slowly (180 mL) at 10° C. to 20° C. Refluxed for 2.0 hrs. Cooled to room temperature and dumped reaction mixture in ice and extracted in ethyl acetate after adjusting the pH to around 10 to 12. Treated the organic layer with brine solution and dried over sodium sulphate. Distilled off under reduced pressure to yield 60.0 gms of 1-(4-chloro-5-nitropyridin-2-yl)-4-methylpiperazine.

### Example 6

### Preparation of 4-chloro-6-(4-methylpiperazin-1-yl)pyridin-3-amine

Charged 100 g of 4-chloro-6-(4-methylpiperazin-1-yl)pyridin-3-amine and 1500 mL acetic acid and 90.0 g iron powder at room temperature. Raised temperature to 75° C. and stirred for 8 hours. Cooled to room temperature and filtered off reaction mixture through celite bed and washed with ethyl acetate. Distilled off mother liquors and diluted in ethyl acetate and washed with saturated sodium bicarbonate solution. Finally organic layer was washed with brine solution and dried over sodium sulphate. Distilled off solvent under reduced pressure to give 4-chloro-6-(4-methylpiperazin-1-yl)pyridin-3-amine (75 gms).

### Example 7

### Preparation of 4-chloro-N-methyl-6-(4-methylpiperazin-1-yl)pyridin-3-amine

Charged 100 g of 4-chloro-6-(4-methylpiperazin-1-yl)pyridin-3-amine, trimethylorthoformate (800 ml) and catalytic amount of trifluoro acetic acid. Refluxed the reaction mass at 100-105° C. for 3 hrs. Distilled the reaction mass and the residue was charged to a solution of lithium aluminium hydride (17.9 g) in tetrahydrofuran at 0° C. to 5° C. Stirred the reaction mass for 60 to 90 mins and then quenched the mass in a mixture of ethyl acetate and process water medium. Adjusted the pH of the reaction mass to 1.0 to 2.0 and extracted the product in ethyl acetate layer. Dried the ethyl acetate layer with sodium sulphate and distilled to get a thick mass which was further used for the next step.

### Example 8

### Preparation of 2-(3,5-bis(trifluoromethyl)phenyl)-N-(4-chloro-6-(4-methylpiperazin-1-yl)pyridin-3-yl)-N,2-dimethylpropanamide

Charged toluene (300 ml) to the residue of Example 7 and cooled to 0° C. to 5° C., charged diisopropylethylamine (64.5 g) to the above clear solution. Charged 2-(3,5-bis(trifluoromethyl)phenyl)-2-methylpropanoyl chloride (158.9 g) dropwise to the above reaction mass and continued stirring for 30 mins at 0° C. to 5° C. Slowly raised the temperature of the reaction mass to 105° C. to 115° C. and continued the reflux for one hr. Cooled to room temperature and quenched the reaction mass in process water and extracted the product in toluene. Dried the toluene layer with anhydrous sodium sulphate and distilled to get a thick residue which on trituration with diisopropylether resulted in solid. Yield 120 gms of 2-(3,5-bis(trifluoromethyl)phenyl)-N-(4-chloro-6-(4-methylpiperazin-1-yl)pyridin-3-yl)-N,2-dimethylpropanamide.

### Example 9

### Preparation of 2-(3,5-bis(trifluoromethyl)phenyl)-N,2-dimethyl-N-(6-(4-methylpiperazin-1-yl)-4-(o-tolyl)pyridin-3-yl)propanamide

Charged 2-(3,5-bis(trifluoromethyl)phenyl)-N-(4-chloro-6-(4-methylpiperazin-1-yl)pyridin-3-yl)-N,2-dimethylpropanamide, (100 gms), 450 ml toluene and 150 ml 2N sodium carbonate solution, 10 gms tetrakis(triphenylphosphine) palladium (0) and O-tolyl boronic acid (105 gms). Refluxed the reaction mass for 6 to 8 hrs and then quenched the reaction mass in crushed ice and separated the layers. Distilled the toluene layer completely and dissolved the residue in diisopropyl ether. Charged concentrated hydrochloric acid to acidic pH and distilled the diisopropyl ether completely to get a free solid which was again suspended in diisopropyl ether. Stirred for 30 mins at room temp and then filtered. Suction dried to yield 60.0 gms of 2-(3,5-bis(trifluoromethyl)phenyl)-N,2-dimethyl-N-(6-(4-methylpiperazin-1-yl)-4-(o-tolyl)pyridin-3-yl)propanamide dihydrochloride.

Scheme III Experiments/Examples

### Example 10

### Preparation of 2-hydroxy-4-methoxy-5-nitro pyridine(4-methoxy-5-nitropyridin-2-ol)

Charged ammonia gas at −70° C. to a solution of THF (1800 ml). After achieving 1.0 Kg, stopped the gas flow. Charged potassium tert butoxide (182 gms). Raised the temperature to −30° C., charged a solution of 4-methoxy-3-nitro pyridine (100 g) dissolved in THF (250 ml) and tert butyl hydro peroxide (144 ml) to the above mixture at −30 to −25° C. After addition, continued the reaction for 1.0 hrs. Saturated ammonium chloride solution (450 ml) was cautiously added and the mixture allowed to warm to room temperature. The ammonia was evaporated and the residue diluted with water (500 mL). The resulting solid was collected, washed with water and dried to give the title compound (90.0 g)

### Example 11

### Preparation of 2-chloro-4-methoxy-5-nitro pyridine

Charged 2-hydroxy-4-methoxy-5-nitro pyridine (100 g) and N, N-dimethylformamide (500 ml) at 0° C.- to 5° C. Added slowly phosphorus oxy chloride (270 g) at 0° C. to 5° C. Raised the temperature to ambient temperature over two hrs and further raised the temperature to 50-60° C. Stirred for 15 to 45 mins and then cooled to ambient temperature. Quenched the reaction mass in crushed ice (3.5 kg) and stirred at 0° C. to 5° C. for 30 to 45 mins. Then, neutralized the reaction mass with saturated sodium carbonate solution to get pH between 6.9-7.1. Filtered the isolated solid and washed it with water. Suction dried well and dried at below 45° C. to get 65 g of 2-chloro-4-methoxy-5-nitro pyridine.

### Example 12

### Preparation of 1-(4-methoxy-5-nitropyridin-2-yl)-4-methylpiperazine

Charged 2-chloro-4-methoxy-5-nitro pyridine (100 g) N-methyl piperazine (175 mL) and tetrahydrofuran (1200 mL) and stirred for 10 hrs at room temperature. Charged reaction mixture in 720 mL of water and extracted in ethyl acetate. Organic layer washed with brine solution and treated with sodium sulphate. Distilled off organic layer under reduced pressure and crystallisation with di isopropyl ether gave the title compound 115.0 gms.

### Example 13

### Preparation of 2-(4-methylpiperazin-1-yl)-5-nitropyridin-4-ol

Charged 1-(4-methoxy-5-nitropyridin-2-yl)-4-methylpiperazine (100 g), 38% HBr in acetic acid (500 mL) and acetic acid (1000 mL). Stirred for 8 hrs at 105° C. Cooled to room temperature and filtered the solid and washed with 100 mL acetic acid. Dried the solid at 70° C. for 10 hrs to give 145 g of the title compound.

### Example 14

### Preparation of 1-(4-chloro-5-nitropyridin-2-yl)-4-methylpiperazine

Charged 2-(4-methylpiperazin-1-yl)-5-nitropyridin-4-ol (100.0 g) and N, N-dimethylformamide (500 ml) at 0° C.- to 5° C. Added slowly phosphorus oxy chloride (270 g)) at 0° C. to 5° C. Raised the temperature to ambient temp in two hrs and further raised the temp to 50 to 60° C. Stirred for 15 to 45 mins and then cooled it to ambient temp. Quenched the reaction mass in crushed ice (3.5 kg) and stirred at 0° C. to 5° C. for 30 to 45 mins. Then, neutralized the reaction mass with saturated sodium carbonate solution to get pH between 6.9 to 7.1. Filtered the isolated solid and washed it with water. Suction dried well and dried it at below 45° C. to get 1-(4-chloro-5-nitropyridin-2-yl)-4-methylpiperazine.

### Example 15

### Preparation of 1-(4-bromo-5-nitropyridin-2-yl)-4-methylpiperazine

Charged 2-(4-methylpiperazin-1-yl)-5-nitropyridin-4-ol (100.0 g) and N,N-dimethylformamide (500 ml) at 0° C.- to 5° C. Added slowly phosphorus oxy bromide (270 g)) at 0° C. to 5° C. Raised the temperature to ambient temperature in two hrs and further raised the temperature to 50 to 60° C. Stirred for 15 to 45 mins and then cooled it to ambient temperature. Quenched the reaction mass in crushed ice (3.5 kg) and stirred at 0° C. to 5° C. for 30 to 45 mins. Then, neutralised the reaction mass with satd sodium carbonate solution to get pH between 6.9-7.1. Filtered the isolated solid and washed it with water. Suction dried well and dried it at below 45° C. to get 1-(4-bromo-5-nitropyridin-2-yl)-4-methylpiperazine.

### Example 16

### Preparation of 1-methyl-4-(5-nitro-4-(o-tolyl)pyridin-2-yl)piperazine

Charged 1-(4-chloro-5-nitropyridin-2-yl)-4-methylpiperazine (100 g), toluene (1 lit.) 2M Na₂CO₃ Solution (436 ml), O-tolyl boronic acid (60 gm.), tetrakis (13 g) were refluxed reaction mass for 2 hrs. Cooled reaction mass to room temperature and quenched reaction mass in a mixture of ethyl acetate and process water medium. Extracted product in ethyl acetate layer. Dried the ethyl acetate layer with sodium sulphate and distilled to get thick mass which on crystallisation with di-isopropyl ether gave 80 g of the of 1-methyl-4-(5-nitro-4-(o-tolyl)pyridin-2-yl)piperazine.

### Example 17

### Preparation of 6-(4-methylpiperazin-1-yl)-4-(o-tolyl)pyridin-3-amine

Charged 1-methyl-4-(5-nitro-4-(o-tolyl)pyridin-2-yl)piperazine (100 g) and 1000 mL acetic acid and 90.0 g iron powder at room temperature. Raised temperature 75° C. and stirred for 8 hours. Cooled to room temperature and filtered off reaction mixture through celite bed and washed with ethyl acetate. Distilled off mother liquors and diluted in ethyl acetate and washed with saturated sodium bicarbonate solution. Finally organic layer washed with brine solution and dried over sodium sulphate and distilled off solvent under reduced pressure to give title 6-(4-methylpiperazin-1-yl)-4-(o-tolyl)pyridin-3-amine 75 gms. Charged concentrated hydrochloric acid to the residue and refluxed for 30 to 60 mins at 90-95° C. Cooled the reaction mass to 20-30° C., charged ethyl acetate and adjusted the reaction mass pH to above 10 and extracted the product in ethyl acetate. Distilled off the ethyl acetate under vacuum to obtain a further purified 6-(4-methylpiperazin-1-yl)-4-(o-tolyl)pyridine-3-amine.

### Example 18

### Preparation of N-methyl-6-(4-methylpiperazin-1-yl)-4-(o-tolyl)pyridin-3-amine

Charged 100 g of 6-(4-methylpiperazin-1-yl)-4-(o-tolyl)pyridin-3-amine (100 g), acetonitrile (1000 ml), diisopropylethylamine (185 ml) and charged ethyl chloroformate (61 ml) at ambient temperature. Stirred for one hr at ambient temperature and raised the temperature to reflux temperature and continue for another one hr. Charged ethyl acetate (500 ml) and process water (500 ml). Stirred and separated the layers. Distilled the ethyl acetate layer to a thick residue and charged tetrahydrofuran (1000 ml). Charged lithium aluminium hydride (54 g) in lot wise at 0 to 5° C. Stirred the reaction mass for 60 to 90 mins and then refluxed for 3 to 4 hrs. Quenched the mass in a mixture of ethyl acetate and process water medium and extracted the product in ethyl acetate layer. Dried the ethyl acetate layer with sodium sulphate and distilled to get a thick mass which was further considered for the next step.

### Example 19

### Preparation of 2-(3,5-bis(trifluoromethyl)phenyl)-N-(4-chloro-6-(4-methylpiperazin-1-yl)pyridin-3-yl)-N,2-dimethylpropanamide

Charged toluene (300 ml) to the residue of Example 18 and cooled to 0 to 5° C., charged diisopropylethylamine (64.5 g) to the above clear solution. Charged 2-(3,5-bis(trifluoromethyl)phenyl)-2-methylpropanoyl chloride (158.9 g) dropwise to the above reaction mass and continued the stirring for 30 mins at 0 to 5° C. Slowly raised the temperature of the reaction mass to 105 to 115° C. and continued the reflux for one hr. Cooled to room temp and quenched the reaction mass in process water and extracted the product in tolune. Dried the toluene layer with anhydrous sodium sulphate and distilled to get a thick residue which on trituration with di isopropyl ether, resulted in solid. Yield 120 gms of the title product.

### Amorphous Netupitant Experiments/Examples

### Example 20

### Preparation of Amorphous Netupitant

Charge netupitant (300 mg) and povidone (300 mg) at 20 to 25° C. Charge methanol (30 ml) and stir at the above temperature range for 30 to 60 mins for dissolution. Distill off the solvent methanol under vacuum at below 50° C. After complete distillation, scratch the solids from the flask and dry the resultant unloaded solid from the flask at 50 to 60° C. under vacuum. Yield: 200 mg. XRD: Amorphous form (FIG. 1).

### Example 21

### Preparation of Amorphous Netupitant

Charge netupitant (300 mg) and acetone (48 ml) at 20 to 25° C. Stir at the above temperature range for 30 to 60 mins for dissolution. Distill off the solvent acetone under vacuum at below 50° C. Cool to 20 to 25° C. Charge dichloromethane (56 ml) and reflux it to 35 to 39° C. In a separate flask, charge povidone (300 mg) and dichloromethane (15 ml). Reflux this mixture to obtain a clear solution and charge this clear solution to the above solution of netupitant in dichloromethane at 30 to 35° C. Stir for 15 to 30 minutes. Distill off the dichloromethane completely. After complete distillation, scratch the solids from the flask and dry the resultant unloaded solid from the flask at 50 to 60° C. under vacuum. Yield: 200 mg. XRD: Amorphous form (FIG. 2).

Although the compositions and methods of the present disclosure have been described with reference to exemplary embodiments thereof, the present disclosure is not limited thereby. Indeed, the exemplary embodiments are implementations of the disclosed compositions and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

What is claimed is:

1. A process of making netupitant or a pharmaceutically acceptable salt thereof comprising reducing a compound of the formula 3H 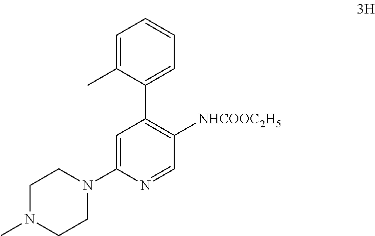
and subsequently coupling the resulting compound with 2-(3,5-bis(trifluoromethyl)phenyl)-2-methylpropanoyl chloride

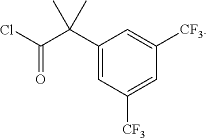 [SMILES: CC(C)(C(=O)Cl)c1cc(C(F)(F)F)cc(C(F)(F)F)c1]

2. Ethyl(6-(4-methylpiperazin-1-yl)-4-(o-tolyl)pyridine-3-yl)carbamate of the formula 3H

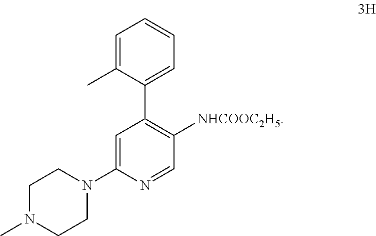

3. A process for making netupitant and pharmaceutically acceptable salts thereof comprising coupling a compound of the formula 2J 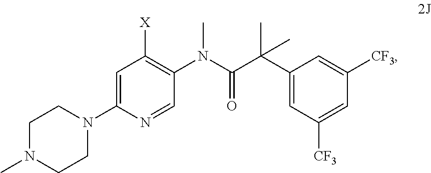
wherein X is an alcohol protecting group, with o-tolyl boronic acid (2L).

4. The process according to claim 3 further comprising forming a hydrochloride of the formula I

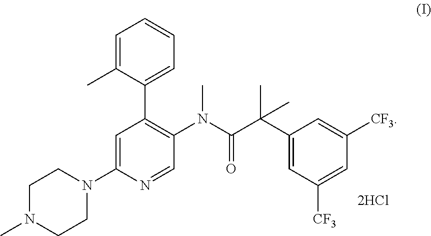 [SMILES: Cc1ccccc1-c1cc(N2CCN(C)CC2)ncc1N(C)C(=O)C(C)(C)c1cc(C(F)(F)F)cc(C(F)(F)F)c1]

5. The process according to claim 3 wherein X is Cl, Br, OCH3, OTf, OBz, or o-pivaloyl.

6. The process according to claim 3 wherein X is Cl.

7. The process according to claim 3 wherein the coupling takes place in the presence of a catalyst and a base.

8. The process according to claim 7 wherein the catalyst is tetrakis (triphenyl phosphine) palladium (0).

9. The process according to claim 7 wherein the base is selected from the group consisting of sodium bicarbonate, potassium bicarbonate, sodium carbonate, potassium carbonate and cesium carbonate.

10. The process according to claim 4, wherein the hydrochloride formation is carried out using hydrochloric acid and an organic solvent.

11. The process according to claim 10 wherein the organic solvent is selected from the group consisting of acetone, isopropanol, methanol, and diisopropyl ether.

12. A process for making netupitant and pharmaceutically acceptable salts thereof comprising coupling of a compound having the formula 2H 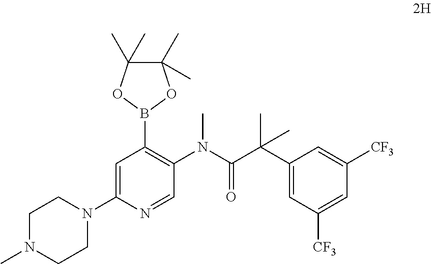 [SMILES: CN1CCN(c2cc(B3OC(C)(C)C(C)(C)O3)c(N(C)C(=O)C(C)(C)c3cc(C(F)(F)F)cc(C(F)(F)F)c3)cn2)CC1]
with a 2-halo substituted toluene.

13. The process according to claim 12 further comprising forming a hydrochloride of the formula I

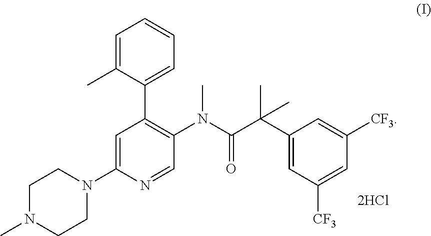 [SMILES: Cc1ccccc1-c1cc(N2CCN(C)CC2)ncc1N(C)C(=O)C(C)(C)c1cc(C(F)(F)F)cc(C(F)(F)F)c1]

14. The process according to claim 13, wherein the hydrochloride formation is carried out using hydrochloric acid and an organic solvent.

15. The process according to claim 12 wherein the coupling takes place in the presence of a catalyst and a base.

16. The process according to claim 15 wherein the catalyst is tetrakis (triphenyl phosphine) palladium (0) or bis(triphenylphosphine)palladium (II) dichloride.

17. The process according to claim 15 wherein the base is selected from the group consisting of sodium bicarbonate, potassium bicarbonate, sodium carbonate, potassium carbonate and cesium carbonate.

18. The process according to claim 14 wherein the organic solvent is selected from acetone, isopropanol, methanol, and diisopropyl ether.

19. The process according to claim 12 wherein the 2-halo substituted toluene is a compound of the formula 2K 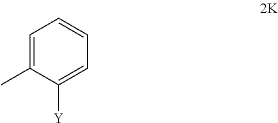
wherein Y is selected form the group consisting of Cl, Br, and I.

20. The process according to claim 12 wherein the 2-halo substituted toluene is 1-iodo-2-methylbenzene.

21. A compound of the formula 2J 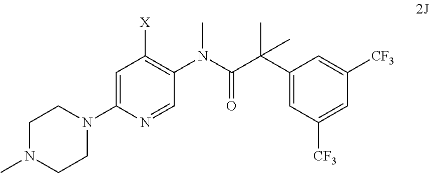
wherein X is Cl, Br, OCH3, OTf, OBz, or o-pivaloyl.

22. A process for making the compound according to claim 21 comprising condensing 4-chloro-6-(4-methylpiperazin-1-yl)pyridin-3-amine with 2-(3,5-bis(trifluoromethyl)phenyl)-2-methylpropanoyl chloride in the presence of an organic base.

23. A compound of the formula 2H

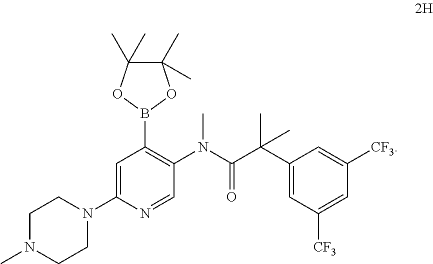 [SMILES: CN1CCN(c2cc(B3OC(C)(C)C(C)(C)O3)c(N(C)C(=O)C(C)(C)c3cc(C(F)(F)F)cc(C(F)(F)F)c3)cn2)CC1]

24. Amorphous netupitant.
